(19) United States
(12) Patent Application Publication
(10) Pub. No.: US 2012/0283290 A1
(43) Pub. Date: Nov. 8, 2012

(54) QUANTITATION OF GL3 IN URINE

(75) Inventor: Sheela Sitaraman, Hoboken, NJ (US)

(73) Assignee: AMICUS THERAPEUTICS INC., Cranbury, NJ (US)

(21) Appl. No.: 13/464,660

(22) Filed: May 4, 2012

Related U.S. Application Data
(60) Provisional application No. 61/483,421, filed on May 6, 2011.

(51) Int. Cl. C12Q 1/34; C07H 15/10; C12Q 1/02; H01J 49/26; A61K 31/445

(52) U.S. Cl. 514/315; 250/282; 435/29; 435/18; 536/17.9

(57) ABSTRACT

The present invention is directed to the quantitation of GL3 in human urine which can be used for the diagnosis of Fabry disease as well as for the assessment of treatment efficacy thereof.

### CROSS-REFERENCE TO RELATED APPLICATION

This application is based on and claims the benefit of U.S. Provisional Application No. 61/483,421, filed May 6, 2011, the entire contents of all of which are incorporated herein by reference.

### FIELD OF THE INVENTION

The field relates to the quantitation of GL3 in human urine. The validated GL-3 assay can be used for the diagnosis of Fabry disease as well as for the assessment of treatment efficacy as shown by a reduction of urine GL-3 upon successful treatment of Fabry Disease.

### BACKGROUND OF THE INVENTION

Fabry disease is caused by mutations in the gene that encodes the lysosomal enzyme a-galactosidase A (or α-Gal A). Deficient activity of this enzyme results in the reduced ability to catabolize certain glycosphingolipids, primarily globotriaosylceramide (GL-3). An inability to catabolize GL-3 results in progressive accumulation of that substrate in the vascular endothelium and in visceral tissues throughout the body. In the kidney, deficient a-Gal A results in accumulation of glycosphingolipid deposits in a variety of cell types, including podocytes, mesangial cells, tubular epithelial cells and endothelial cells (Thurberg et al., 2002).

As GL-3 accumulates, there is progressive damage to the kidneys reflected by proteinuria, glomerulosclerosis, isosthenuria, and azotemia (e.g., elevated serum creatinine levels). Ultimately, progressive accumulation of GL-3 results in end-stage renal disease and renal failure, the most frequent cause of death among males with Fabry disease (Desnick et al., 2001).

It is generally believed that progressive accumulation of microvascular endothelial deposits of GL-3 leads to ischemia and infarction in the kidneys, contributing to kidney damage and disease (Desnick et al., 2001). Studies of enzyme replacement therapy (ERT), an approved treatment for Fabry disease, have demonstrated a reduction in the number of GL-3 inclusions in renal interstitial capillaries, and have also suggested a delay in the progression of renal disease (Banikazemi et al., 2007; Eng et al., 2001; Schiffmann et al., 2007).

Changes in urine GL-3 can be detected over relatively short (several weeks) periods of time, presumably due to a rapid turnover of distal tubule cells (Nadasdy et al., 1994) which contribute a large portion of the GL-3 measured in urine. It has been estimated that approximately 70,000 kidney tubular epithelial cells are shed into the urine every hour (Pryor et al., 2000). The majority of the reduction in urine GL-3 observed in patients treated with enzyme replacement therapy (ERT) appears to occur within the first 6 months, suggesting that urine GL-3 reductions can be measured in this timeframe.

Previously, studies of Fabry patients have failed to consistently correlate disease manifestation and individual symptoms with elevated urinary GL-3. Thus, it was thought that urine GL-3 was of limited value as surrogate marker of Fabry disease (Vedder et al., 2007). There is a need for an accurate, sensitive, and reliable assay to measure urine GL-3 for diagnosis of Fabry disease as well as for monitoring the efficacy of treatment of Fabry disease.

### SUMMARY OF THE INVENTION

Provided is a validated method for the quantitation of GL3 in urine. In one embodiment, the assay measures C22:0 and C24:0 isoforms of GL-3 in human urine. In some embodiments, purified synthetic GL-3 isoforms can be used for assay calibration as well as for spiking samples with known quantities of these isoforms. A method for analytical quantitation of C22:0 and C24:0 in human urine via HPLC with MS/MS detection is provided. The use of the well characterized synthetic reference standards allows quantitation of these GL-3 isoforms to a lower limit of quantitation than previously possible. This urine GL-3 assay can accurately quantify C22:0 and C24:0 isoforms down to a lower limit of quantitation of 1 ng/mL.

In one embodiment, correction for the possibility of irregular shedding of kidney cells is made by measuring GL-3 in urine after a 24 hour collection period. Urine processing can include a sonication step to lyse whole cells, which allows quantification of GL-3 in both the sediment (intact cells) and supernatant (lysed cells and filtered GL-3) fractions of whole urine.

The validated method is preferably applicable to quantitation of GL-3 C22:0 and GL-3 C24:0 isoformS within a nominal range of 1.00 to 200 and with a 200-μL human urine aliquot containing Tween 80. The validated test can be used in both stripped and un-stripped urine. Additionally, samples can be kept frozen at approximately −70° C. prior to analysis.

### DETAILED DESCRIPTION OF THE PREFERRED EMBODIMENT

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The term “Fabry disease” refers to an X-linked inborn error of glycosphingolipid catabolism due to deficient lysosomal α-galactosidase A activity. This defect causes accumulation of globotriaosylceramide (ceramide trihexoside) and related glycosphingolipids in vascular endothelial lysosomes of the heart, kidneys, skin, and other tissues.

The term “atypical Fabry disease” refers to patients with primarily cardiac manifestations of the α-GAL deficiency, namely progressive globotriaosylceramide (GL-3) accumulation in myocardial cells that leads to significant enlargement of the heart, particularly the left ventricle.

A “carrier” is a female who has one X chromosome with a defective α-GAL gene and one X chromosome with the normal gene and in whom X chromosome inactivation of the normal allele is present in one or more cell types. A carrier is often afflicted with Fabry disease.

A “patient” refers to a subject who has been diagnosed with a particular disease. The patient may be human or animal. A “Fabry disease patient” refers to an individual who has been diagnosed with Fabry disease and has a mutated α-GAL as defined further below. Characteristic markers of Fabry disease can occur in male hemizygotes and female carriers with the same prevalence, although females typically are less severely affected.

Human α-galactosidase A (α-GAL) refers to an enzyme encoded by the human Gla gene. The human α-GAL enzyme consists of 429 amino acids and is represented by GenBank Accession No. U78027.

As used herein in one embodiment, the term “mutant α-GAL” includes an α-GAL which has a mutation in the gene encoding α-GAL which may result in the inability of the enzyme to achieve a stable conformation under the conditions normally present in the ER. It may also result it a lower specific enzyme activity. The failure to achieve a stable conformation can result in a substantial amount of the enzyme being degraded, rather than being transported to the lysosome. Such a mutation is sometimes called a “conformational mutant.”

Non-limiting, exemplary α-GAL mutations associated with Fabry disease which result in unstable α-GAL include L32P; N34S; T41I; M51K; E59K; E66Q; I91T; A97V; R100K; R112C; R112H; F113L; T141L; A143T; G144V; S148N; A156V; L166V; D170V; C172Y; G183D; P205T; Y207C; Y207S; N215S; A228P; S235C; D244N; P259R; N263S; N264A; G272S; S276G; Q279E; Q279K; Q279H; M284T; W287C; I289F; M296I; M296V; L300P; R301Q; V316E; N320Y; G325D; G328A; R342Q; E358A; E358K; R363c; R363H; G370S; and P409A.

“Deficient α-GAL activity” refers to α-GAL activity in cells from a patient which is below the normal range as compared (using the same methods) to the activity in normal individuals not having or suspected of having Fabry or any other disease (especially a blood disease).

As used herein, the terms “enhance α-GAL activity” or “increase α-GAL activity” refer to increasing the amount of α-GAL that adopts a stable conformation in a cell contacted with a pharmacological chaperone specific for the α-GAL, relative to the amount in a cell (preferably of the same cell-type or the same cell, e.g., at an earlier time) not contacted with the pharmacological chaperone specific for the α-GAL. This term can also refer to increasing the trafficking of α-GAL to the lysosome in a cell contacted with a pharmacological chaperone specific for the α-GAL, relative to the trafficking of α-GAL not contacted with the pharmacological chaperone specific for the protein. These terms refer to both wild-type and mutant α-GAL. In one embodiment, the increase in the amount of α-GAL in the cell is measured by measuring the hydrolysis of an artificial substrate in lysates from cells that have been treated with the SPC. An increase in hydrolysis is indicative of increased α-GAL activity.

The term “α-GAL activity” refers to the normal physiological function of a wild-type α-GAL in a cell. For example, α-GAL activity includes hydrolysis of GL-3.

The term “GL-3” or “globotriaosylceramide,” also known as ceramide trihexoside, CTH or Gb3, means a glycosphingolipid having the following structure:

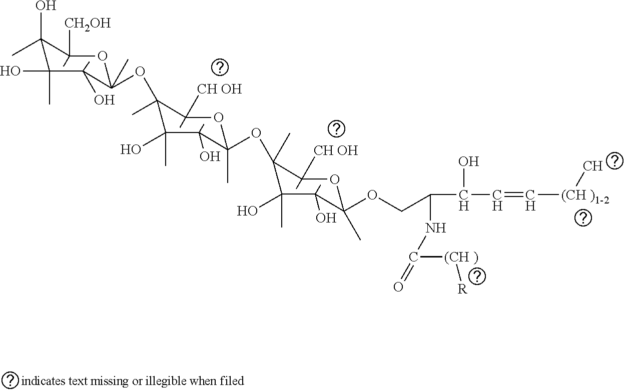

wherein R is a saturated or unsaturated lipid chain.

GL-3 C22:0 isoform means the isoform of GL-3 wherein R is a 20 carbon long saturated lipid chain, thus resulting in a 22 carbon long saturated fatty acid.

GL-3 C24:0 isoform meats the isoform of GL-3 wherein R is a 22 carbon long saturated lipid chain, thus resulting in a 24 carbon long saturated fatty acid. These GL3 isoforms also mean the definitions given in various research articles (some of which are incorporated by reference).

“Quantitation” means to measure or determine the quantity of a molecule or substance.

The term “substantially pure” means that the target molecule does not contain any related-substance impurity at a concentration of greater than about 10% by weight, preferably less than about 1% by weight, and most preferably less than about 0.1% by weight.

The term “substantially pure synthetic” means a substantially pure molecule that is derived through any chemical synthesis or a biological process that has been modified resulting in molecules of higher purity than is possible without modification.

“Calibration” means comparison of a sample of known magnitude or correctness made or set with one sample, device, material, molecule or assay and measurement made in a similar way. The sample, device, material, molecule or assay with the known or assigned correctness is typically called a reference or standard.

The term “treatment” means to therapeutically intervene in the development of a disease in a subject showing a symptom of this disease.

A “surrogate marker” or “surrogate clinical marker” of Fabry disease refers to the abnormal presence of, increased levels of, abnormal absence of, or decreased levels of a biomarker or symptom that is associated with Fabry disease (but is not associated with a healthy individual), and which is a reliable indicator of Fabry disease either alone or in combination with other abnormal markers or symptoms of Fabry disease.

The term “therapeutic effect” or “therapeutic response” may be any response that a user (e.g., a clinician) will recognize as an effective response to the therapy, including improvements in any of the foregoing symptoms and surrogate clinical markers. A therapeutic response may be any response that a user (e.g., a clinician) will recognize as an effective response to the therapy, including improvements in the foregoing symptoms and surrogate clinical markers. Thus, a therapeutic response will generally be an amelioration of one or more symptoms or markers of a disease or disorder, such as those described above.

The term “contents of a patient's cells” means the components of a mammalian cell that are bounded by a cell membrane. These contents may be inside the cell or, after the integrity of the cell membrane is compromised, outside the normal confines of the cell membrane. These contents may also be in a form that has been purified, such as for example, removal of non-soluble materials, membranes, DNA or other contents that are not necessary for a particular assay. The level of purification can vary, and includes substantial pure patients GL3 enzyme and recombinant patient GL3.

The term “α-galactosidase A substrate” means any material, substance or chemical that the enzyme a-galactosidase A acts upon or interacts with.

“Exogenous α-galactosidase A substrate” refers to any α-galactosidase A substrate that is externally introduced to the patient's cells.

The term “matrix” means any biological sample such as for example plasma, serum, blood, cellular contents, bodily secretions, urine etc.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The assessment of urine GL-3 using the analytically validated assay includes important technical improvements over currently employed urine GL-3 assays. The validated assay can measure the kidney-predominant GL-3 isoforms, C22:0 and C24:0, to identify kidney-specific deterioration, Collection of urine over an extended time period corrects for the possibility of irregular shedding of kidney cells. In one embodiment, urine is collected over a 24 hour period. Shedded cells that are present in urine can be lysed by a variety of methods to quantitatively release GL3. Urine processing can include a sonication step to lyse whole cells, which allows quantification of GL-3 in both the sediment (intact cells) and supernatant (lysed cells and filtered GL-3) fractions of whole urine. A lower limit of quantitation for the GL-3 isoforms (1 ng/mL) is possible with this validated method.

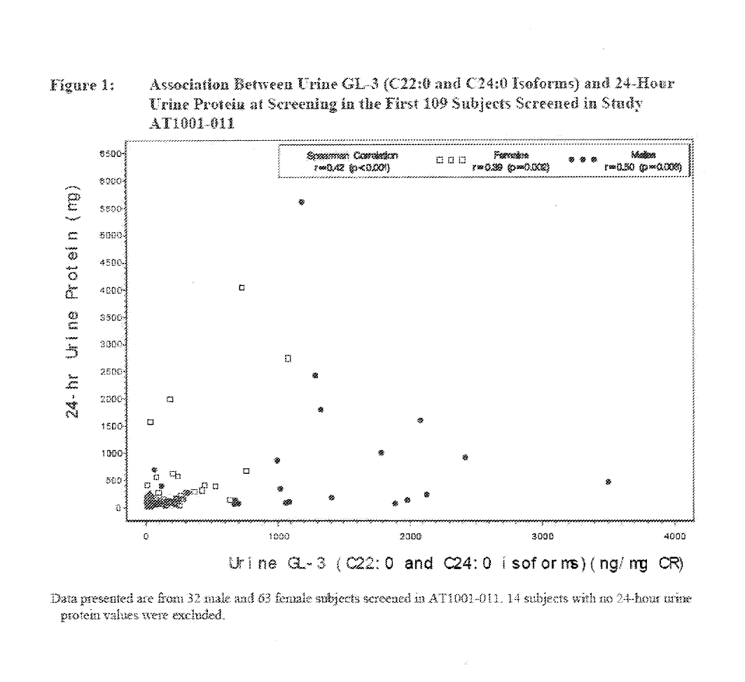

Prior studies by clinical investigators have been unable to definitively demonstrate a direct link between urine GL-3 and clinical outcome. Using this validated urine GL-3 assay for the C22:0 and C24:0 isoforms an association was observed between increased urine GL-3 and increased 24-hour urine protein (Spearman correlation; r=0.42, p<0.001), see FIG. 1. A number of studies have shown that elevated 24-hour urine protein is predictive of an increased rate of decline in renal function in Fabry disease (Schiffmann, Warnock et al. 2009; West, Nicholls et al. 2009).

The GL-3 present in urine is thought to arise from three principle sources, shed or lysed tubular cells (majority), other shed or lysed cells (e.g., podocytes, urinary tract), and from filtration from plasma. Urine GL-3 is comprised of both kidney GL-3 excreted in the form of sloughed renal tubular cells and systemic GL-3 that is filtered through the glomerulus and excreted in the urine.

When measured appropriately, quantification of GL-3 isoforms derived predominantly from kidney cells shed into the urine offers a sensitive measure of GL-3 that broadly integrates the substrate accumulation status of a multitude of pathophysiologically relevant cells from both kidneys in males and females. The C22:0 and C24:0 isoforms are the most predominant isoforms in kidney tissue (Sweeley and Klionsky 1963; Mårtensson 1966); and the cells likely contributing the majority of these GL-3 isoforms in urine (tubular cells) are pathophysiologically relevant to Fabry nephropathy. In plasma the most abundant GL-3 isoforms are C16:0 (55%) and C24:1 (13%) (Nelson, Roddy et al. 2004; Mills, Morris et al. 2005; Chrastina, Martincová et al. 2007).

Measurement of urine GL-3 may vary based on changes in plasma GL-3, which does not correlate with kidney disease especially in females (Gupta, Ries et al. 2005; Bekri, Lidove et al. 2006). The analytically validated assay measures C22:0 and C24:0 which are the predominant GL-3 isoforms in kidney tissue. Importantly, C16:0—the predominant GL-3 isoform in plasma—need not be quantified in the urine assay.

This integrated sampling of cells is especially important in female Fabry patients, where assessment of a finite amount of cells (e.g. from a kidney biopsy) is susceptible to uneven sampling of healthy and diseased cells due to mosacism and inherent patchiness from random X chromosome inactivation (Gubler. Lenoir et al. 1978; Migeon 2008; Valbuena, Carvalho et al. 2008).

Based on the Phase 2 studies of the migalastat HCl treatment of Fabry disease, the C22:0 and C24:0 isoforms collectively represent approximately 50% of the total urine GL-3 measured. In addition, the pattern of the individual GL-3 isoforms in urine is similar to that of total GL-3 indicating that one or more individual isoforms can be representative of total urine GL-3. Determination of urine GL-3 based on the sum of the C22:0 and C24:0 isoforms alone was highly correlated with total urine GL-3 hast on the sum of six isoforms (r=0.99).

Other analyses of Fabry patients have failed to correlate disease manifestation and individual symptoms with elevated urinary GL-3. Thus, previously, it was concluded that urine GL-3 was of limited value as surrogate marker of Fabry disease (Vedder et al., 2007).

This analytically validated assay has been successfully used to determine urine GL-3 levels in healthy individuals and establish a normal range. See Table 1. Using this assay an upper limit of normal has been defined in healthy individuals (33.8 ng/mg creatinine) for urine GL-3 as measured by a combination of C22:0 and C24:0,

TABLE 1
Healthy Normals (M + F) Levels of C22:0 and C24:0 in ng/mL

| | | | Combined C220 |
|---|---|---|---|
| | | | and C240 |
| Sample ID | C220 (ng/mL) | C240 (ng/mL) | Value(ng/mL) |
| 1 | 4.41 | 3.9 | 8.31 |
| 2 | 3.84 | 3.02 | 6.86 |
| 3 | 14.1 | 11.1 | 25.2 |
| 4 | 7.79 | 5.34 | 13.13 |
| 5 | 8.97 | 9.35 | 18.32 |
| 6 | 17.9 | 13.9 | 31.8 |
| 7 | 6.46 | 5.24 | 11.7 |
| 8 | 4.33 | 3.08 | 7.41 |
| 9 | 3.6 | 2.9 | 6.5 |
| 10 | 5.37 | 3.74 | 9.11 |
| 11 | 14.1 | 9.65 | 23.75 |
| 12 | 1.16 | 1.16 | 2.32 |
| 13 | 3.66 | 2.93 | 6.59 |
| 14 | 7.67 | 6.08 | 13.75 |
| 15 | 22.4 | 15.9 | 38.3 |
| 16 | 8.95 | 6.8 | 15.75 |
| 17 | 5.03 | 3.67 | 8.7 |
| 18 | 11.7 | 7.61 | 19.31 |
| 19 | 3.12 | 2.52 | 5.64 |
| 20 | 9.37 | 7.91 | 17.28 |
| 21 | 5.97 | 3.82 | 9.79 |
| 22 | 22 | 15.1 | 37.1 |
| 23 | 20.8 | 15.5 | 36.3 |
| 24 | 11.6 | 10.9 | 22.5 |
| 25 | 22 | 11.2 | 33.2 |
| 26 | 7.46 | 7.62 | 15.08 |
| 27 | 1.58 | 1.35 | 2.93 |
| 28 | 10.1 | 8.12 | 18.22 |
| 29 | 4.26 | 3.1 | 7.36 |
| 30 | 3.05 | 2.23 | 5.28 |
| 31 | 9.52 | 6.7 | 16.22 |
| 32 | 14.2 | 9.43 | 23.63 |
| 33 | 8.42 | 6.3 | 14.72 |
| 34 | 10.1 | 8.62 | 18.72 |
| 35 | 8.43 | 6.89 | 15.32 |
| 36 | 1.53 | 1.21 | 2.74 |
| 37 | 4.72 | 4.34 | 9.06 |
| 38 | 9.25 | 7.51 | 16.76 |
| | | Mean | 15.6 |
| | | S.D. | 9.9 |

### EXAMPLES

### Example I

### Measurement of Urine GL-3

In this embodiment of the invention, an LC-MS/MS method was used to measure GL-3 in human urine. Six GL3 isoforms were measured using a reference standard of biological origin. The specific isoforms measured were C16:0, C18:0, C20:0, C22:0, C24:0 and C24:1 The C17 isoform of GL-3 was also measured as an internal standard in the assay and is a non-naturally occurring isoform. The data can be reported as Total GL-3 which is a summation of all measured and quantifiable isoforms. The pattern of the individual GL-3 isoforms in urine is similar to that of total GL-3, indicating that one or more individual isoforms could be used to represent total GL-3.

The GL-3 reference material of biological origin contained a mixture of several isoforms in addition to the six being measured. Because well characterized isolated reference standards are not currently available for individual isoforms of GL-3, another embodiment of the method uses two synthesized reference standards. Given that the pattern of the two most abundant isoforms are similar to that of total GL-3, reference standards for these two isoforms are used. The specific isoforms measured are C22:0 and C24:0.

### Example II

### Measurement of GL3 in Human Urine Using C22:0 and C24:0 GL-3 Isoforms

A 200-μL matrix aliquot was fortified with 20 μL of 100 ng/mL C17-CTH internal standard working solution. Analytes were isolated through liquid-liquid extraction using about 2:1 chloroform/methanol, v/v. The eluate was evaporated. Evaporation can be performed by any known method such as under a nitrogen stream at approximately 45° C. The remaining residue was reconstituted with 200 μL of 0.2 mM sodium acetate in methanol The final extract was analyzed via HPLC and MS/MS detection using positive ion electrospray.

Eight calibration standards were analyzed in duplicate over the nominal concentration range of 1.00 to 200 ng/mL for C22:0 and C24:0. A linear, 1/concentration squared weighted, least-squares regression algorithm was used to plot the peak area ratio of the appropriate analyte to its internal standard versus concentration. The average correlation coefficient from six standard curves was >0.990.

The lower limit of quantitation was the lowest non-zero concentration level that was quantified with acceptable accuracy and precision. For the validation of one embodiment, the lower limit of quantitation was nominally 1.00 ng/mL for C22:0 and C24:0.

Precision and accuracy were evaluated by analyzing quality control pools prepared at 1.00, 2.00, 5.00, 15.0, 40.0, and 150 ng/mL for C22:0 and C24:0 in stripped urine, and 19.5 and 154 ng/mL for C22:0 and 18.4 and 153 ng/mL for C24:0 in un-stripped urine.

Precision can be expressed as the percent coefficient of variation (% CV) of each pool. Accuracy was measured as the percent difference from theoretical.

Table 2 shows the analysis of patient urine GL-3 Using the validated assay. Table 1 shows mutations that cause Fabry disease. Mutations such as R342Q are known to cause high urine GL-3. However, previous assays have failed to show this in a consistent fashion. Table 1 shows that, using the validated assay, eight out of eight patients tested showed elevated urine GL-3.

TABLE 2

| | URINE GL-3 | |
|---|---|---|
| | LEVEL (FOLD | SUBJECT |
| MUTATION | OVER NORMAL) | (M or F) |
| R112H | 0.6 | F |
| | 1.2 | F |
| | 3.5 | M |
| | 2.8 | M |
| | 0.7 | F |
| | 4.4 | F |
| N215S | 1.44/0.6 | M |
| | 0.5 | M |
| | 0.7 | F |
| | 0.4 | M |
| | 0.8 | M |
| | 0.6 | F |
| | 0.8 | F |
| | 0.72 | M |
| | 1.2 | M |
| | 0.97 | M |
| | 0.89 | M |
| | 1.9 | M |
| | 0.6 | F |
| | 0.5 | F |
| | 2.6 | F |
| | 0.7 | F |
| | 1.7/2.0 | F |
| R301Q | 3.76 | F |
| | 22 | F |
| | 11 | F |
| | 5 | F |
| | 2.4 | F |
| | 38 | M |
| | 2.4 | F |
| R342Q | 4 | F |
| | 30.8 | M |
| | 8 | F |
| | 27.8 | M |
| | 19.9 | M |
| | 19 | F |
| | 4.7 | F |
| | 34.9 | M |
| H125P | 71.6 | M |
| | 5.4 | F |
| | 1.8 | F |
| | 2 | F |
| | 5.8 | F |

Quantitation of GL3 in human urine has been disclosed in U.S. Provisional Patent Application No. 61/483,421, filed May 6, 201 which is incorporated herein by reference in its entirety and for all purposes.

All other documents referred to herein are incorporated by reference into the present application as though fully set forth herein.

The following articles are incorporated by reference:
(2002). Advisory Committee Briefing Document on BLA STN 103979/0: Recombinant human α-Galactosidase for Fabry's disease.
(2003a). Advisory Committee Briefing Document on BLA STN 103977/0: agalsidase alfa for Fabry's Disease.
(2003b). European Public Assessment Report—Scientific Discussions on Fabrazyme, In European Medicines Agency (The Netherlands: Genzyme Europe B.V.).
Banikazerni, M., Bultas, J., Waldek, S., Wilcox, W. R., Whitley, C. B., McDonald, M., Finkel, R., Packman, S., Bichet, D. G., Warnock, D. G., and Desnick, R. J. (2007). Agalsidase-beta therapy for advanced Fabry disease: a randomized trial. Ann Intern Med 146, 77-86 Epub 2006 December 2018.
Desnick, R., Ioarmou, Y., and Eng, C. (2001). α-Galactosidase A deficiency: Fabry disease., In The Metabolic and Molecular Bases of inherited Disease, S. C R, B. A L, S. W S, and V. D, eds. (New York: McGraw-Hill).
Eng, C. M., Guffon, N., Wilcox, W. R., Germain, D. P., Lee, P., Waldek, S., Caplan, L, Linthorst, G. E., and Desnick, R. J. (2001). Safety and efficacy of recombinant human alphagalactosidase A—replacement therapy in Fabry's disease. N Engl J Med 345, 9-16.
Howie, A. J., Ferreira, M. A. S., and Adu, D. (2001). Prognostic value of simple measurement of chronic damage in renal biopsy specimens. Nephrol Dial Transplant 16, 1163-1169.
Kitagawa, I., Ishige, N., Suzuki, K., Owada, M., Ohashi, T., Kobayashi, M., Eto, V., Tanaka, A., Mills, K., Winchester, B., and Keutzer, J. (2005). Non-invasive screening method for Fabry disease by measuring globotriaosylceramide in whole urine samples using tandem mass spectrometry. Molecular Genetics and Metabolism 85, 196-202.
Lidove, O., Joly, D., Barbey, F., Bekri, S., Alexandra, J. F., Peigne, V., Jaussaud, R., and Papo, T. (2007). Clinical results of enzyme replacement therapy in Fabry disease: a comprehensive review of literature. International Journal of Clinical Practice 61, 293-302.
Najatian, B., Gubler, M.-C., Whitley, C., and Mauer, M. (2009). 95. Podocyte injury and GL-3 accumulation are progressive in Fabry disease. Molecular Genetics and Metabolism 96, S33-S33.
Ries, M., Gupta, S., Moore, D. F., Sachdev, V., Quirk, J. M., Murray, a J., Rosing, D. R., Robinson, C., Schaefer, E., Gal, A., et al. (2005). Pediatric Fabry Disease. Pediatrics 115, e344-355.
Schiffmann, R., Askari, H., Timmons, M., Robinson, C., Benko, W., Brady, R. O., and Ries, M. (2007). Weekly enzyme replacement therapy may slow decline of renal function in patients with Fabry disease who are on long-term biweekly dosing. J Am Soc Nephroi 18, 1576-1583 Epub 2007 April 1574.
Thurberg, B. L., Remike, H., Colvin, R. B., Dikman, S., Gordon, R. E., Collins, A. B., Desniek, R. J., and O'Callaghan, M. (2002). Globotriaosylceramide accumulation in the Fabry kidney is cleared from multiple cell types after enzyme replacement therapy. Kidney Int 62, 1933-1946.
Valbuena, C., Carvalho, E., Bustorff, M., Ganhão, M., Reivas, Nogueira, R., Carneiro, F., and Oliveira, J. (2008). Kidney biopsy findings in heterozygous Fabry disease females with early nephropathy. Virchows Archly 453, 329-338.
Vedder, A., Linthorst, G. van Breernen, M., Groener, J., Bemelman, F., Strijiand, A., Mannens, M., Aerts, S., Hollak, C. (2007). The Dutch Fabry cohort: Diversity of clinical manifestations and Gb3 levels. J. Inherited Metabolic Disease 30 (1), 68-78.

What is claimed is:

1. A method for measuring GL3 in a mammal comprising quantitation of GL3 C22:00 isoform in a sample from said mammal.

2. A method for measuring GL3 in a mammal comprising quantitation of GL3 C24:00 isoform in a sample from said mammal.

3. A method for measuring GL3 in a mammal comprising quantitation of GL3 C22:00 isoform and C24:00 isoform in a sample from said mammal.

4. The method according to claim 1, wherein the sample is urine.

5. The method of claim 2, wherein the sample is urine.

6. The method according to claim 3, wherein the sample is urine.

7. The method according to claim 1, wherein the GL3 isoforms are quantitated using HPLC and MS/MS detection.

8. The method of claim 2, wherein the GL3 isoforms are quantitated using HPLC and MS/MS detection.

9. The method according to claim 3, wherein the GL3 isoforms are quantitated using HPLC and MS/MS detection.

10. A method for measuring GL3 in a mammal comprising quantitation of GL3 C22:00 isoform and GL3 C24:00 isoform in the urine from said mammal wherein the GL3 isoforms are quantitated using HPLC and MS/MS detection.

11. A method for treating a patient with Fabry disease, comprising determining whether there is a change in the amounts of GL3 C22:00 isoform and GL3 C24:00 isoform in the urine from said patient wherein the GL3 isoforms are quantitated with an assay using HPLC and MS/MS detection.

12. The method according to claim 11, wherein the assay uses substantially pure synthetic GL3 C22:00 isoform and substantially pure synthetic GL3 C24:00 isoform for calibration of the assay.

13. The method according to claim 11, wherein the treatment further comprises administering a pharmacological chaperone to the patient.

14. The method according to claim 11, wherein the pharmacological chaperone is an inhibitor of α-galactosidase A.

15. A method of monitoring the response of a patient to the treatment of Fabry disease, the method comprising assaying the level of GL3 in a first sample from the patient before treatment and comparing it with the level of GL3 from a second sample from the patient after treatment, wherein the assay measures the amounts of GL3 C22:00 isoform and GL3 C24:00 isoform in the samples from said patient, wherein a reduction in GL3 level in the second sample as compared to the GL3 level in the first sample indicates a therapeutic effect.

16. A method of monitoring the response of a patient to the treatment of Fabry disease with a pharmacological chaperone, the method comprising assaying the level of GL3 in the urine from the patient before treatment and comparing it with the level of GL3 in the urine from the patient after treatment, wherein the assay measures the amounts of GL3 C22:00 isoform and GL3 C24:00 isoform in the urine from said patient, wherein a reduction in GL3 level in the urine compared to the GL3 level before treatment with the chaperone indicates a therapeutic effect.

17. A method of monitoring the response of a patient to the treatment of Fabry disease with a pharmacological chaperone, the method comprising assaying the level of GL3 in the urine from the patient before treatment and comparing it with the level of G13 in the urine from the patient after treatment, wherein the assay quantitates the amounts of GL3 C22:00 isoform and GL3 C24:00 isoform in the urine from said patient using HPLC and MS/MS detection, wherein a reduction in GL3 level in the urine compared to the GL3 level before treatment with the chaperone indicates a therapeutic effect.

18. The method according to claim 16 wherein the pharmacological chaperone is 1-deoxygalactonojirimycin or migalastat hydrochloride.

19. A method of monitoring the response of a patient with Fabry disease to treatment with 1-deoxygalactonojirimycin, the method comprising assaying the level of GL3 in the urine from the patient before treatment and comparing it with the level of GL3 in the urine from the patient after treatment, wherein the assay measures the amounts of GL3 C22:00 isoform and GL3 C24:00 isoform in the urine from said patient, wherein a reduction in GL3 level in the urine compared to the GL3 level before treatment with 1-deoxygalactonojirimycin indicates a therapeutic effect.

20. A method for determining if a patient with Fabry disease will respond to treatment with a pharmacological chaperone, comprising quantitating in vitro the amount of GL3 in the contents of a patient's cells before the contents are contacted with the chaperone and comparing to the amount of GL3 after contacting the contents with the chaperone, wherein the assay uses synthetic GL3 C22:00 isoform and synthetic GL3 C24:00 isoform.

21. A composition for the measurement of GL3 in a mammal comprising substantially pure synthetic GL3 C22:00 isoform and substantially pure synthetic GL3 C24:00 isoform.

22. A composition for the measurement of GL3 in a mammal comprising a sample from a mammal containing substantially pure synthetic GL3 C22:00 isoform and/or substantially pure synthetic GL3 C24:00 isoform.

23. The method according to claim 22 wherein the sample is urine.

24. A composition for monitoring the response of a mammal to the treatment of Fabry disease comprising substantially pure synthetic GL3 C22:00 isoform and/or substantially pure synthetic GL3 C24:00 isoform.

25. The composition of claim 24 further comprising urine.

26. A composition for determining if a patient with Fabry disease will respond to treatment with a pharmacological chaperone, comprising a patient cell in contact with substantially pure synthetic GL3 C22:00 isofbrm and/or substantially pure synthetic GL3 C24:00 isoform

27. The method according to claim 1, wherein the sample is urine, plasma, kidney cells or skin cells.

28. The method according to claim 2, wherein the sample is urine, plasma, kidney cells or skin cells.

29. The method according to claim 3, wherein the sample is urine, plasma, kidney cells or skin cells.

30. A method according to claim 1, wherein the mammal is a carrier of Fabry disease.

31. A method according to claim 2, wherein the mammal is a carrier of Fabry disease.

32. A method according to claim 3, wherein the mammal is a carrier of Fabry disease.

33. A method according to claim 1, wherein the patient has a missense mutation in α-Gal A and wherein DGJ is administered in a dosage regimen comprising either 150 mg every other day or escalating doses of 25, 100 and then 250 mg b.i.d. over 6 weeks followed by 50 mg/day.

34. A method according to claim 2, wherein the patient has a missense mutation in α-Gal A and wherein DGJ is administered in a dosage regimen comprising either 150 mg every other day or escalating doses of 25, 100 and then 250 mg b.i.d. over 6 weeks followed by 50 mg/day.

35. A method according to claim 3, wherein the patient has a missense mutatiOn in α-Gal A and wherein DGJ is administered in a dosage regimen comprising either 150 mg every other day or escalating doses of 25, 100 and then 250 mg b.i.d, over 6 weeks followed by 50 mg/day.

36. A method according to claim 15 wherein DGJ is administered to the patient in an amount of 150 mg every other day.

37. A method according to claim 16 wherein DGJ is administered to the patient in an amount of 150 trig every other day.

38. A method according to claim 17 wherein DGJ is administered to the patient in an amount of 150 mg every other day.

39. A method according to claim 18 wherein DGJ is administered to the patient in an amount of 150 mg every other day.

40. A method according to claim 19 wherein DGJ is administered to the patient in an amount of 150 mg every other day.

41. A method according to claim 16 wherein GL3 levels are monitored at baseline and after 3 months of treatment.

42. A method according to claim 17 wherein GL3 levels are monitored at baseline and after 3 months of treatment.

43. A method according to claim 18 wherein GL3 levels are monitored at baseline and after 3 months of treatment.

44. A method according to claim 19 wherein GL3 levels are monitored at baseline and after 3 months of treatment.

45. A method according to claim 41, wherein GL3 levels are further monitored after 6 months of treatment.

46. A method of monitoring treatment of Fabry disease, comprising administering to the patient a pharmacological chaperone at a dose of about 75-225 mg once every other day and assaying the level of GL3 wherein the assay measures GL3 C22:00 isoform and GL3 C24:00 isoform.

47. The method according to claim 46 wherein the pharmacological chaperone is 1-deoxygalactonojirimycin.

48. The method according to claim 47 wherein the assay uses substantially pure synthetic GL3 C22:00 isoform and/or substantially pure synthetic GL3 C24:00 isoform.

49. A method of monitoring treatment of Fabry disease, comprising administering to a patient a pharmacological chaperone at a dose of about 75-225 mg once every other day and administering recombinant human α-galactosidase A, wherein the method further comprises assaying the level of GL3 by measuring GL3 C22:00 isoform and GL3 C24:00 isoform.

50. The method according to claim 49, wherein exogenous α-galactosidase A substrate is added to the assay.

51. A method of identifying pharmacological chaperones, the assay comprising: contacting α-galactosidase A with a substrate and a test molecule; measuring GL3 by measuring GL3 C22:00 isoform and/or GL3 C24:00 isoform; determining the enhancement of α-alactosidase A by the test molecule.

52. The method according to claim 51, wherein the α-galactosidase A has a mutation.

53. The method according to claim 52, wherein the mutant α-galactosidase A is from a human.

54. The method according to claim 51, wherein the assay is conducted at two different pH's.

55. The method according to claim 52, wherein the assay is conducted at two different pH's.

56. The method according to claim 53, wherein the assay is conducted at two different pH's.

57. A kit for measuring GL3 in a mammal comprising GL3 C22:00 isoform or GL3 C24:00
